(19) United States
(12) Patent Application Publication
(10) Pub. No.: US 2015/0216540 A1
(43) Pub. Date: Aug. 6, 2015

(54) Trans pars interarticularis operation for releasing of the adhesions in lumbar spinal reoperations

(71) Applicant: Ashkan Khajoo, Tehran (IR)

(72) Inventor: Ashkan Khajoo, Tehran (IR)

(21) Appl. No.: 14/545,238

(22) Filed: Apr. 13, 2015

(51) Int. Cl. A61B 17/16; A61B 17/56

(52) U.S. Cl. CPC A61B 17/1671; A61B 17/56; A61B 2017/564; A61B 2019/4884

(57) ABSTRACT

This invention proposes releasing adhesions and pressure on nerve roots of spinal cord by directly drilling a hole in pars interarticularis and accessing the nerve root laterally from outside of vertebra, which does not cause any damage to the spinal cord and its dural (dural is a lining which covers the cord).

### BACKGROUND OF THE INVENTION

A common method to release pressure on nerve roots, which are caused by previous spinal surgery, is to use a special device and enter the spinal canal from upper or lower part of the vertebras and via the inner part of canal which is not manipulated previously and slowly and meticulously access the adhesion areas on the cord. The next step is to remove the adhesions on the nerve root which is very time consuming and has the risk of spinal cord injury such as dural tear (dural is a lining which covers the cord).

### SUMMARY OF THE INVENTION

This invention proposes a different method of accessing nerve root and spinal cord are that has adhesions and is under pressure, which does not cause any damage to the spinal cord and its dural (dural is a lining which covers the cord). Using this invention, a surgeon accesses the nerve root directly and laterally and from outer side of the vertebral foramen. This is done by directly drilling hole in the pars interarticularis right above the nerve root. This creates a hole; from which the nerve root can be accessed without a need to enter the spinal canal and risk of injuring the spinal cord or dural tear. A special adhesion removal device can then be used to remove adhesions on the nerve via this hole. One advantage of this method is that there is no spinal cord injury which is a common complication of classic approach of releasing

### BRIEF DESCRIPTION OF THE DRAWINGS

FIG. 1 is a schematic diagram of cross section of spine and spinal cord. This diagram shows the foramen, nerve root, the interarticularis and the vertebra bone.

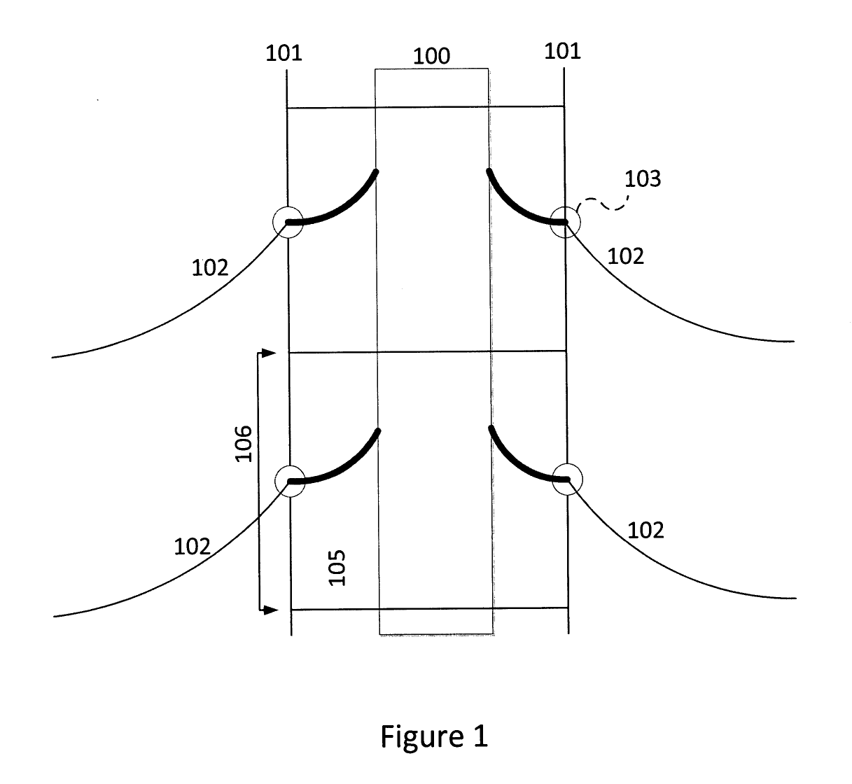

### DETAILED DESCRIPTION OF THE PREFERRED EMBODIMENTS

There are 24 vertebras (106) with hole which form the spinal canal (105) in a human body. Spinal cord (100) passes through the spinal canal (105). Nerves are attached to the spinal cord via nerve roots (102). These nerves separate and exit at each level of vertebra from a foramen (103) in the vertebra (106) and go to the muscles.

In routine methods of spinal surgery, spinal canal (105) is opened and instruments are inserted in the spinal canal (105) via the joints between vertebras (106). An example is operations to correct canal stenosis. After most spinal surgeries, fibrosis may form and cause adhesions of nerve roots (102) (to the foramen and press the nerve roots against the foramen (103) from which nerve root exits. This pressure would cause pain on that root.

To reduce the pain, the pressure should be removed from the nerve root (102). A common method is to use a special device and enter the spinal canal from upper or lower part of the vertebras (106) and via the inner part of canal (105) which is not manipulated previously and slowly and meticulously access the adhesion areas on the cord. The next step is to remove the adhesions on the nerve root (102) which is very time consuming and has the risk of spinal cord injury such as dural tear (dural is a lining which covers the cord).

One embodiment of this invention is to access the nerve root directly by accessing the nerve root laterally and from outer side of the vertebral foramen. An embodiment of the invention creates access to the nerve root by directly drilling the pars interarticularis (103) right above the nerve root. This creates a hole; from which the nerve root can be accessed without a need to enter the spinal canal and risk of injuring the spinal cord or dural tear. A special adhesion removal device can then be used to remove adhesions on the nerve via this hole. Then the involved segment can be fixed with proper instrumentation and fusion.

One advantage of this method is that there is no spinal cord injury which is a common complication of classic approach of releasing adhesions and pressure from nerve roots.

In one embodiment of this invention the holes is not filled and is left open to allow the pressure to be release continuously.

In an embodiment of the invention the affected vertebra is screwed to the neighboring vertebra, to create a better support for the spinal cord and prevent undesired movement.

This surgery technique has been performed on 41 patients with 100% success rate.

Any variations of the above teaching are also intended to be covered by this patent application.

1. A method of operation to release the pressure and adhesions of spinal nerve root, said method comprising:
Accessing the lateral and outside of the vertebral foramen;
drilling a small hole in the pars interarticularis;
separating the nerve from the vertebral;
releasing pressure and adhesions on said nerve root.

2. The method of claim 1 wherein said hole is drilled via a precision drill, wherein said precision drill is stopped before it hits said nerve root.

3. The method of claim 1 wherein said hole is left uncovered after said operation.

4. The method of claim 1 wherein said hole is covered after said operation.

5. The method of claim 1 wherein said vertebral is attached to a neighbor vertebral via brace and screws.

6. The method of claim 1 wherein said operation is performed on patients with previous spinal operation.

7. The method of claim 1 wherein said hole is drilled directly above the said nerve root.

8. The method of claim 1 wherein said hole is drilled some small distance away from the said nerve root.

9. The method of claim 1 wherein said pars interarticularis is fixed with proper instrumentation and fusion.

10. The method of claim 1 where in no damage is made to spinal cord.
